(19) United States
(12) Patent Application Publication
(10) Pub. No.: US 2022/0354677 A1
(43) Pub. Date: Nov. 10, 2022

(54) DELIVERY OF AMNION TISSUE GRAFT WITH TEMPORARY SUPPORT

(71) Applicant: Peytant Solutions, Inc., Plymouth, MN (US)

(72) Inventors: John Schorgl, Chanhassen, MN (US); Robert E. Kohler, Lake Elmo, MN (US)

(21) Appl. No.: 17/732,963

(22) Filed: Apr. 29, 2022

Related U.S. Application Data
(60) Provisional application No. 63/186,464, filed on May 10, 2021.

(51) Int. Cl. A61F 2/958; A61B 17/12

(52) U.S. Cl. CPC A61F 2/958; A61B 17/12118; A61B 2017/1205; A61F 2002/823

(57) ABSTRACT

An amnion tissue delivery system for delivering amnion tissue into a location within a patient is described. The location within a patient may be a tube, vessel, lumen, orifice, fistula, aneurysm, or chamber. The amnion tissue delivery system employs a catheter and extendable element, such as a removable stent-like structure, in a configuration such that extension of the extendable section deploys the amnion tissue at the desired location. A method for delivery of amnion tissue within a patient is also described. The method includes placing the catheter within the patient using a minimally invasive, endoluminal and/or extraluminal procedure to position tissue, followed by deploying the extendable element to deliver the amnion tissue at the desired location.

### CROSS REFERENCE TO RELATED APPLICATIONS

This application claims priority to copending provisional patent application 63/186,464 to Schorgl et al., entitled “Delivery of Amnion Tissue Graft with Temporary Support,” incorporated herein by reference.

### FIELD OF THE INVENTION

This disclosure relates generally to a medical device and, in particular, to a catheter delivery system for the delivery of tissue to a location inside the body during surgery.

### BRIEF DESCRIPTION OF THE DRAWINGS

FIG. 1 is a schematic depiction of amnion graft and delivery deployment system assembly with amnion graft preloaded on an example delivery deployment stent.

FIG. 2 is a schematic depiction of amnion graft detached from the delivery deployment stent after the stent has been slid out from inside the graft.

FIG. 3 is a schematic depiction of amnion graft being loaded on a deflated balloon of a balloon catheter.

FIG. 4A is a schematic depiction of amnion graft loaded on a balloon catheter with balloon expanded inside graft.

FIG. 4B is a schematic depiction of the amnion graft and balloon catheter shown in FIG. 4A, wherein the balloon is deflated and removed from inside the graft.

FIG. 5 is a schematic depiction of amnion tissue patch rolled circumferentially to make a tube for delivery using the devices and methods disclosed herein.

### SUMMARY OF THE INVENTION

In a first aspect, the invention pertains to an amnion tissue delivery system comprising a catheter designed for placement into a location within a patient, an extendable element mounted on the catheter, and a section of amnion-based tissue mounted over the extendable section, such as a removable stent-like structure, in a configuration such that extension of the extendable section deploys the amnion tissue at a position selected within the patient. With respect to the amnion tissue delivery system, the desired location within the patient can be a tube, vessel, lumen, orifice, fistula, aneurysm, or chamber. The extendable element can comprise metal, metal alloy, plastic, bioresorbable polymer, or combinations thereof, and wherein the section of amnion-based tissue is associated for delivery along with a biologic on or in the tissue, a polymer component, a metal alloy component or a combination thereof. The extendable element can comprise an expandable balloon or an extending mechanical element, such as a stent. The stent can be balloon extended or self extended. In some embodiments, the self-extending mechanical element comprises a shape memory metal configured to transition to the extended configuration. In an alternate embodiment, the extending mechanical element comprises a shape memory metal configured to be mechanically expanded to deploy the amnion, and configured to transition to the pre-extended configuration with a temperature greater than body temperature.

In the amnion tissue delivery system, the amnion tissue can have a biological glue on its outer surface to adhere the tissue to the patient upon deployment. In some embodiments, the amnion tissue is associated with a removable stent structure that releases with a thermal bond release mechanism. In additional or alternative embodiments, the extendable element comprises a release mechanism, such as clasps, gripping claws or an engagement surface that is configured to temporarily hold the amnion tissue against the patient tissue.

For use in the amnion tissue delivery system, the amnion tissue can be processed to be decellularized. In some embodiments, the amnion-based tissue can comprise a graft, patch, barrier, coating, covering, filler, or plug that is temporarily mounted on the expandable delivery mechanism

In further aspects, the invention pertains to a method for the delivery of amnion tissue within a patient using the amnion tissue delivery system as described herein. The method can comprise:

placing the catheter of the amnion tissue delivery system within the patient using a minimally invasive, endoluminal, extraluminal, and/or endoscopic procedure to position the tissue at a desired location; and

deploying the extendable element to deliver the amnion-based tissue at the selected location in the patient. The method can further comprise removing the catheter and extendable element to leave the amnion-based tissue within the patient.

### DETAILED DESCRIPTION OF THE INVENTION

Placental tissue comprises two major membrane components, the amnion and the chorion with the amnion being interior to the chorion in relation to the amniotic sac that encloses a mammalian, e.g., human, embryo. As described herein, the amnion layer of the amniotic sac can be separated from the chorion to be used directly. The side of the separated amnion tissue with the epithelium layer is generally referred to as the front side while the side with the extracellular matrix composition is generally referred as the back side. Alternatively, placental tissue, which contains both the amnion layer and the chorion layer, can be used to make a tissue for delivery as described herein. The side of the placental tissue with the amnion layer is generally referred to as the front side while the side with the chorion layer is generally referred as the back side. As used herein, the term “amnion tissue” refers to the amnion layer of the amniotic sac, a portion thereof, or any material including or derived from the amnion layer. The term “placenta tissue” refers to any material comprising a composition from the amniotic sac, such as the amnion layer, the chorion layer, portions thereof or combinations thereof.

In some embodiments, the front layer of the placental tissue facing toward a vessel surface in a patient is a generally suitable configuration, although other configurations may also be suitable. Amnion tissue is particularly desirable for the applications described herein because amnion tissue can be expanded generally without damaging the tissue, and such expansion is generally associated with deployment of a delivery device as described herein. While the discussion here focuses on the delivery of amnion tissue left in a vessel without continued stent support, the corresponding use of amnion tissue as a cover for a permanent stent is described in U.S. Pat. No. 9,205,177 B2 to Schorgl et al., entitled “Stents Modified With Material Comprising Amnion Tissue and Corresponding Processes,” incorporated herein by reference. The amnion-based tissue can be decellularized (free of viable cells), reconstituted, or substantially intact with viable cells.

While cryopreservation can be used for preserving cell cultures for some cell types and has been proposed for tissue storage, cryopreservation can change the mechanical properties of the tissue in undesirable ways, for example, with the loss of at least some of the elasticity. For tissues with viable cells, cryopreservation can lead to cell death. Therefore, in some embodiments, it can be desirable to store the tissue at temperatures above −55° C., in further embodiments greater than about −30° C. and in other embodiments from about −20 to about 10° C. It may be desirable to use storage techniques for tissue with maintained viable cells. A person of ordinary skill in the art will recognize that additional ranges of storage temperature within the explicit ranges above are contemplated and are within the present disclosure.

Human amnion has been shown to assist with healing or treatment of a variety of medical conditions. Placement of the amnion inside the body using minimally invasive techniques is challenging. As described herein, a catheter based method can be used to deliver a amnion graft, patch, barrier, coating, covering, filler, plug temporarily mounted on a stent like or an expandable delivery mechanism in and/or on the lumen, orifice, chamber, fistula, aneurysm or the like. After the amnion is delivered, the delivery mechanism is removed and the amnion is left in the body. The expandable delivery mechanism can be removed within seconds, minutes, hours or days after the amnion is delivered. The desired location using the amnion tissue delivery system within the patient can be a tube, vessel, lumen, orifice, fistula, aneurysm, or chamber.

The delivery procedure can comprise the following steps:

1. Amnion tissue is mounted on a catheter delivery system with a delivery mechanism or stent like deployment system.
2. Delivery system is minimally invasive, endoluminal, extraluminal, and/or endoscopic, catheter based introducer system
3. Delivery system positions the amnion to the desired location inside the body (tube, vessel, lumen, chamber).
4. The delivery system deployment (alloy, plastic, bioabsorbable) deliveries graft material (optionally with a biologic, polymer, alloy).
5. The graft material can be released using an actuation based on mechanical structure, magnetic disengagement, thermal bond release, glue release based on degradation of the glue under physiological conditions.
6. Delivery mechanism or stent deployment system can be removed leaving graft material in place.
7. Delivery mechanism or stent deployment system release mechanism (clasps, grips claws), release the graft material
8. The graft material can be secured to the patient by an adhesive or mechanical bond

The steps outline above could also be used without the delivery mechanism or stent deployment system. In this case the amnion graft would be loaded directly around a balloon catheter. When the balloon is inflated the amnion graft would open up and be positioned against the lumen wall. The balloon would be deflated and removed leaving the amnion graft or patch inside the body.

Stents have many issues of breakage, graft delamination, thrombus, infection, scar tissue, granuloma, foreign body reaction, migration. Amnion has been shown to provide several benefits for healing or to reduce inflammation. The system and methods described herein utilize the therapeutic benefits of amnion without using a permanent structure and avoids complications associated with a permanent implant.

The novel concept described herein is the ability to deploy a device percutaneously, through natural orifice catheter, and/or endoscopically that can deliver an amnion graft material inside the human body. The delivery system will be retrievable such that only the amnion graft and securing element will remain inside the body.

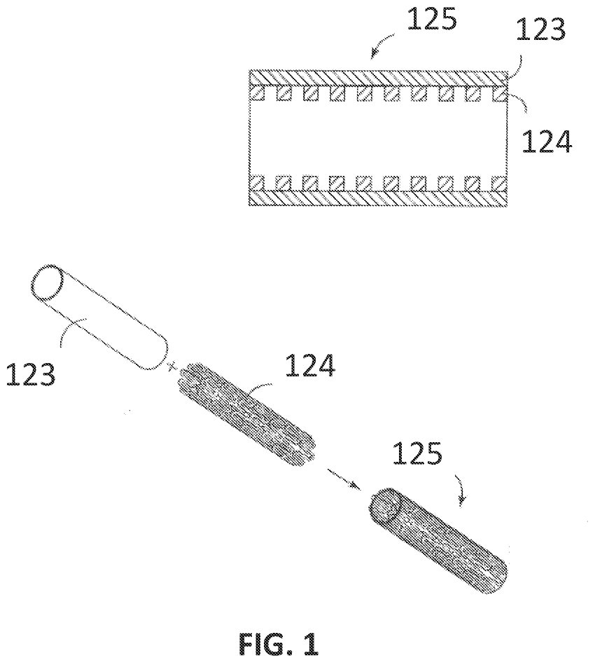

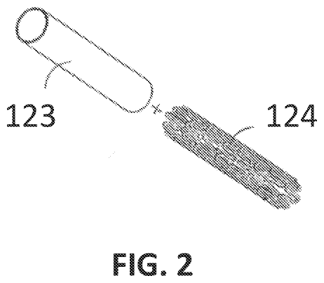

FIG. 1 shows the amnion graft (123) is preloaded on the delivery deployment stent (124). The amnion graft and delivery deployment system assembly (125) are attached to a catheter based introducer system. This system is used to position the amnion graft and delivery deployment system assembly (125) inside the body to the target location. After positioning in the body FIG. 2 shows the amnion graft (123) detached from the delivery deployment stent (124) by sliding the stent out from the inside of the amnion graft (123). An adhesive on the outside of the amnion graft (123) or a mechanical barb may be used to secure the amnion graft (123) inside the body.

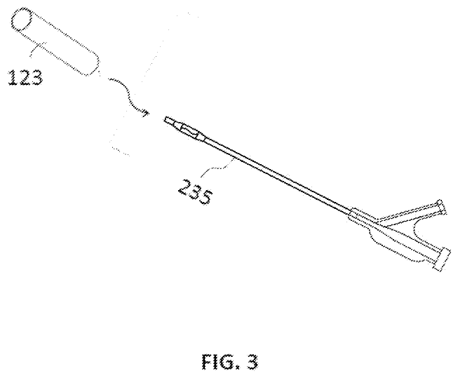

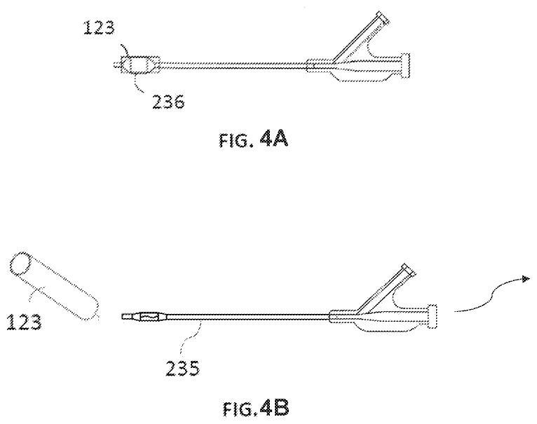

A second method for delivery of the amnion graft would be performed without the use of the delivery deployment stent (124). FIG. 3 illustrates a balloon catheter delivery based system. FIG. 3 Represents the amnion graft (123) loaded on the balloon catheter (235) and assembled on the deflated balloon. When the balloon (236) shown in FIG. 4A is inflated the amnion graft (123) is expanded inside the lumen. The balloon is then deflated and removed leaving the amnion graft (123) inside the body, FIG. 4B.

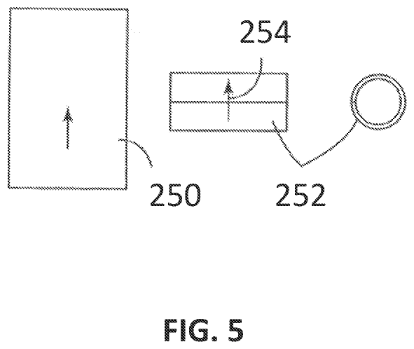

FIG. 5 represents a third embodiment of the present invention that delivers an amnion patch (250). In this invention the amnion patch is rolled circumferentially to make a tubular shape (252). The tubular shape (252) is delivered inside the body using similar delivery techniques describe above.

The embodiments above are intended to be illustrative and not limiting. Additional embodiments are within the claims. In addition, although the present invention has been described with reference to particular embodiments, those skilled in the art will recognize that changes can be made in form and detail without departing from the spirit and scope of the invention. Any incorporation by reference of documents above is limited such that no subject matter is incorporated that is contrary to the explicit disclosure herein. To the extent that specific structures, compositions and/or processes are described herein with components, elements, ingredients or other partitions, it is to be understand that the disclosure herein covers the specific embodiments, embodiments comprising the specific components, elements, ingredients, other partitions or combinations thereof as well as embodiments consisting essentially of such specific components, ingredients or other partitions or combinations thereof that can include additional features that do not change the fundamental nature of the subject matter, as suggested in the discussion, unless otherwise specifically indicated.

What is claimed is:

1. An amnion tissue delivery system comprising a catheter designed for placement into a location within a patient, an extendable element mounted on the catheter, and a section of amnion-based tissue mounted over the extendable section in a configuration such that extension of the extendable section deploys the amnion tissue at a position selected within the patient.

2. The amnion tissue delivery system of claim 1 wherein the desired location within the patient is a tube, vessel, lumen, orifice, fistula, aneurysm, or chamber.

3. The amnion tissue delivery system of claim 1 wherein the extendable element comprises metal, metal alloy, plastic, bioresorbable polymer, or combinations thereof, and wherein the section of amnion-based tissue is associated for delivery with a biologic, a polymer, a metal alloy or a combination thereof.

4. The amnion tissue delivery system of claim 1 wherein the extendable element comprises a balloon or a self-extending mechanical element.

5. The amnion tissue delivery system of claim 4 wherein the self-extending mechanical element comprises a shape memory metal configured to transition to the extended configuration.

6. The amnion tissue delivery system of claim 1 wherein the amnion tissue has a biological glue on its outer surface to adhere the tissue to the patient upon deployment.

7. The amnion tissue delivery system of claim 1 wherein the amnion tissue is associated with a removable stent structure that releases with a thermal bond release mechanism.

8. The amnion tissue delivery system of claim 1 wherein the extendable element comprises a release mechanism that is configured to temporarily hold the amnion tissue against the patient tissue prior to or simultaneously with release of the tissue from the extendable element.

9. The amnion tissue delivery system of claim 8 wherein the release mechanism comprises clasps, gripping claws or an engagement surface.

10. The amnion tissue delivery system of claim 1 wherein the amnion tissue is processed to be decellularized.

11. The amnion tissue delivery system of claim 1 wherein the amnion-based tissue comprises a graft, patch, barrier, coating, covering, filler, or plug that is temporarily mounted on the expandable delivery mechanism

12. The amnion tissue delivery system of claim 1 wherein the amnion-based tissue forms a tubular shaped element.

13. The amnion tissue delivery system of claim 12 wherein the tubular shaped element is rolled amnion patch.

14. A method for the delivery of amnion tissue within a patient using the amnion tissue delivery system of claim 1, the method comprising:
placing the catheter within the patient using a minimally invasive, endoluminal, extraluminal, and/or endoscopic procedure to position the tissue at a desired location; and
deploying the extendable element to deliver the amnion-based tissue at the selected location in the patient; and
removing the extendable element from the patient to leave the amnion-based tissue within the patent.
